(19) United States
(12) Patent Application Publication
(10) Pub. No.: US 2025/0006307 A1
(43) Pub. Date: Jan. 2, 2025

(54) Identification of Changing Pathways or Disease Indicators Through Cluster Analysis

(71) Applicant: DH Technologies Development Pte. Ltd., Singapore (SG)

(72) Inventor: Stephen A. TATE, Barrie (CA)

(21) Appl. No.: 18/690,571

(22) PCT Filed: Sep. 6, 2022
(86) PCT No.: PCT/IB2022/058384

§ 371 Date: Mar. 8, 2024

Related U.S. Application Data
(60) Provisional application No. 63/260,975, filed on Sep. 8, 2021.

(51) Int. Cl. G16B 40/10; G16B 40/30; H01J 49/00

(52) U.S. Cl. CPC G16B 40/10; G16B 40/30; H01J 49/0036

(57) ABSTRACT

A mass range is fragmented and mass analyzed using n samples, producing measurements in n dimensions. Two clustering algorithms are applied to the measurements, producing two sets of clusters, and compounds in the sets are identified. Two or more compounds found in a cluster of both sets are identified. The two or more compounds are compared to groups of compounds related to a biological process to identify a group that includes the two or more compounds. An additional compound is selected from the group. The two sets are reanalyzed to identify the compound in the sets. A co-occurrence matrix is calculated that quantifies the co-occurrence of the compound and each of the two or more compounds in the sets. If no co-occurrence quantity for the compound and each of the two or more compounds in the matrix is below a threshold, the two or more compounds are verified.

### RELATED APPLICATIONS

This application claims the benefit of U.S. Provisional Patent Application Ser. No. 63/260,975, filed on Sep. 8, 2021, the content of which is incorporated by reference herein in its entirety.

### INTRODUCTION

The teachings herein relate to determining whether or not clustering or co-clustering as applied to n-dimensional mass spectrometry data produces meaningful results. More particularly the teachings herein relate to systems and methods for verifying a group of compounds identified through co-clustering. This is done by locating the group in an interacting group of a cell, a biological pathway, or a disease indicator. At least one additional compound in the interacting group of a cell, biological pathway, or disease indicator not part of the group is identified. Reanalysis of the clustering data is performed using the additional compound and co-occurrence of the additional compound with the compounds of the group is quantified. The group is verified or not verified based on this quantification of the co-occurrence.

The systems and methods herein can be performed in conjunction with a processor, controller, or computer system, such as the computer system of FIG. 1.

### Multiple Clustering Algorithms and Biological Significance

Processing mass spectrometry data for biological pathways or disease indicators typically starts with the identification and then quantification of a large number of different compounds. The resulting data is then used to statistically show the biological pathway or disease indicator as a molecular fingerprint. Even though this has brought about significant discoveries, the ability to discern real differences at the biology level is confounded by the noise and lack of simplified methods for the processing of the data.

More specifically, processing mass spectrometry data for biological pathways or disease indicators typically starts with a large biomarker study, for example. This requires large-scale quantitative analysis. This is an analysis of hundreds of thousands of proteins or peptides. Note that the following discussion is related to proteins or peptides. However, the systems and methods described herein can also be applied to any type of compound or metabolite.

From this biomarker study, what would appear to be many different individual components are identified. The mass spectrometry analysis for the study begins, for example, with precursor ion to product ion transitions. A transition is related to a peptide and a peptide is related to a protein. A transition should be doing exactly what its peptide is doing. It is not always the case that a peptide is doing what its protein is doing. The peptide could be a modified form or spliced variant. However, for simplicity, it can be assumed that most peptides are doing what their protein is doing.

Each transition, peptide, or protein is thought of as an independent mass spectrometry measurement. However, for biological pathways or disease indicators, proteins are not independent. Proteins can be part of an interacting group, for example. They are noncovalently associated and bound together in a cell. At the even higher pathway or disease level, the protein interaction is described.

FIG. 2 is an exemplary diagram 200 showing a biological pathway that includes five proteins, upon which embodiments of the present teachings may be implemented. In diagram 200, protein A links to protein B, which links to protein C. Protein B also links to protein D, which links back to protein A. Protein C, in turn, links to protein E. A disease indicator may have a similar pattern with a different set of proteins, for example.

For some time, clustering algorithms have been used to find the relationships between proteins from mass spectrometry measurements. These relationships are then compared to known interacting groups, pathways, or disease indicators to determine if a biological process was detected. U.S. Pat. Nos. 7,587,285, 8,180,581, and 9,442,887 (hereinafter the “'285 Patent,” the “'581 Patent,” and the “'887 Patent,” respectively), for example, all describe a clustering algorithm referred to as principal component analysis with principal component variable grouping (PCA-PCVG) that is used to group or cluster mass spectrometry data and are herein incorporated by reference. Another popular clustering algorithm used to group or cluster mass spectrometry data is referred to as K-means clustering.

FIG. 3 is an exemplary series 300 of plots showing how PCA-PCVG is used to identify compounds from mass spectrometry data, upon which embodiments of the present teachings may be implemented. A spectrum 310 and XICs 320 from a region of interest are used to fill in a matrix 330 of intensities for all masses in a mass range (detector time of arrival), including zeroes. Principal component analysis (PCA) 340 is performed on this data. A clustering technique called PCVG 350 is used to group related features in n-dimensional principal component (PC) space. Reconstructing a spectrum from a subset of each group generates a spectrum 360 for a specific compound. One or more compounds are found for each group. Reconstructing a spectrum from all features simply results in the original unprocessed spectrum 310.

Identifying specific compounds from product ion mass spectra involves comparing the spectra to a library of spectra for known compounds or comparing the spectra to compound fragments predicted from a compound database. Identifying proteins can be done in a bottom-up method or a top-down method. In a bottom-up method, proteins are first digested into peptides before mass spectrometry analysis. In a top-down method, intact proteins are analyzed.

FIG. 4 is an exemplary PCA-PCVG plot 400 showing the location of a protein group in PC space that includes the proteins of FIG. 2 in relation to other protein groups or clusters, upon which embodiments of the present teachings may be implemented. Protein group 410 of plot 400 includes proteins A, B, C, D, and E of the biological pathway of FIG. 2. As described above, proteins A, B, C, D, and E are found from product ion spectra using a library of spectra for known proteins or comparing the spectra to product ions predicted from a protein database.

The K-means clustering algorithm, for example, groups similar values (log 2 fold change values or area values across samples), within a larger population of data, into smaller groups. This algorithm requires a clustering seed for the number of groups. The algorithm randomly picks K values from the observation values to serve as cluster seeds. Then, all of the observation values are grouped into K clusters based on their proximity to each of the cluster seed values. For each cluster group, a mean observation value is determined. Then, all of the observation values are re-grouped into K clusters based on their proximity to each of the cluster group mean seed values. The algorithm iterates through the cluster group mean seed values and proximity determination until there is no shift in cluster group assignment for each observation.

FIG. 5 is an exemplary K-means clustering algorithm plot 500 showing the location of a protein group that includes the proteins of FIGS. 2 and 4 in relation to other protein groups or clusters, upon which embodiments of the present teachings may be implemented. Protein group 510 of plot 500 includes proteins A, B, C, D, and E of the biological pathway of FIG. 2.

Ronan et at, “Avoiding common pitfalls when clustering biological data,” Sci Signal, 2016 Jun. 14; 9(432), re6 (hereinafter the “Ronan Paper”) has identified three problems with the use of conventional clustering algorithms, such as PCA-PCVG and K-means. The first problem is the high dimensionality of the data used to identify a biological pathway or disease indicator. The Ronan paper addresses this problem by reducing the dimensionality of the data.

The second problem is the failure to consider results from more than one clustering algorithm. The Ronan paper addresses this problem by using multiple clustering algorithms or multiple parameters for a single clustering algorithm (ensemble clustering) and co-clustering the output produced to identify proteins or genes which are changing similarly in experimental data sets. Using this method, the Ronan Paper found that known interactors had a much higher tendency to cluster together than non-interactors.

FIG. 6 is an exemplary diagram 600 showing that the intersection of a group from the clustering algorithm of FIG. 4 and a group from the clustering algorithm of FIG. 5 produces a co-clustered protein group that includes the proteins of the biological pathway of FIGS. 2, upon which embodiments of the present teachings may be implemented. Specifically, diagram 600 shows that the intersection of protein group 410 of the PCA-PCVG clustering algorithm and protein group 510 of the K-means clustering algorithm includes proteins A, B, C, D, and E of the biological pathway of FIG. 2.

One method of showing how similar proteins are clustered across two or more different clustering algorithms is to show their distance on a cluster dendrogram. In diagram 600, proteins A, B, C, D, and E are all shown at the same level or with the same distance in clustering dendrogram 610. This shows graphically that proteins A, B, C, D, and E are all part of a stable cluster. Dendrogram 610 is found from the co-occurrence matrix (not shown).

The third problem is the difficulty in determining whether clustering or co-clustering produces meaningful results. The Ronan paper describes that most successful analyses often result from combining clustering with previous biological understanding of the system. The Ronan paper, however, does not describe how previous biological understanding of the system should be combined with clustering. Instead, it simply suggests that the process of attaching biological meaning to clusters be done with statistical analysis. In particular, the Ronan paper cautions against failing to account for any increase in false positives when co-clustering is performed.

As a result, additional systems and methods are needed for combining clustering and co-clustering with additional biological information in order to improve the identification of biological pathways or disease indicators from mass spectrometry data.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components. In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 amu is scanned or stepped across an entire mass range. A product ion spectrum is produced for each 1 amu precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 amu, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the m/z of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

A system, method, and computer program product are disclosed for verifying compounds of a group detected by co-clustering are related to a biological process. The system includes a mass spectrometer and a processor. The mass spectrometer fragments and mass analyzes a mass range using at least m measurements for each sample of at least n different samples, producing at least m measurements in n dimensions.

The processor applies a first clustering algorithm and a second clustering algorithm to the n-dimensional measurements, producing a first set of clusters and a second set of clusters, respectively, and identifies compounds in the first set and the second set. The processor selects a first cluster from the first set and a second cluster from the second set that includes the same two or more compounds. The processor compares the two or more compounds to one or more groups of compounds related to a biological process to identify at least one group of the one or more groups that includes the two or more compounds.

The processor selects at least one compound other than the two or more compounds in the at least one group. The processor reanalyzes the first set and the second set to identify the at least one compound in the first set and the second set. The processor calculates a co-occurrence matrix that quantifies the co-occurrence of the at least one compound and each of the two or more compounds for both the first set and the second set. If no co-occurrence quantity of the at least one compound and each of the two or more compounds in the co-occurrence matrix is below a predetermined threshold, the processor verifies that the two or more compounds are related to a biological process.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary diagram showing a biological pathway that includes five proteins, upon which embodiments of the present teachings may be implemented.

FIG. 3 is an exemplary series of plots showing how PCA-PCVG is used to identify compounds from mass spectrometry data, upon which embodiments of the present teachings may be implemented.

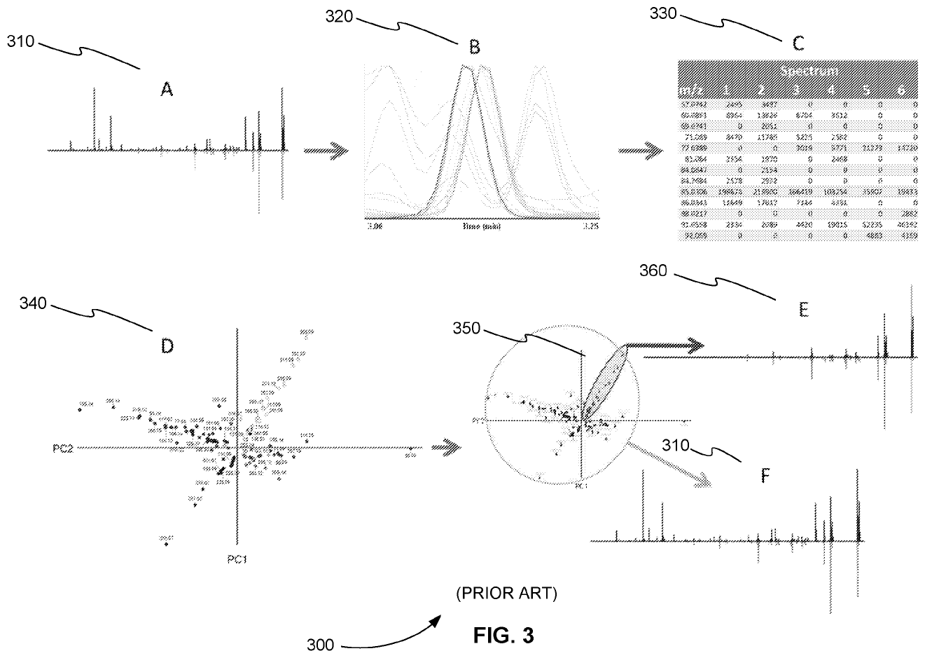

FIG. 4 is an exemplary PCA-PCVG plot showing the location of a protein group in PC space that includes the proteins of FIG. 2 in relation to other protein groups or clusters, upon which embodiments of the present teachings may be implemented.

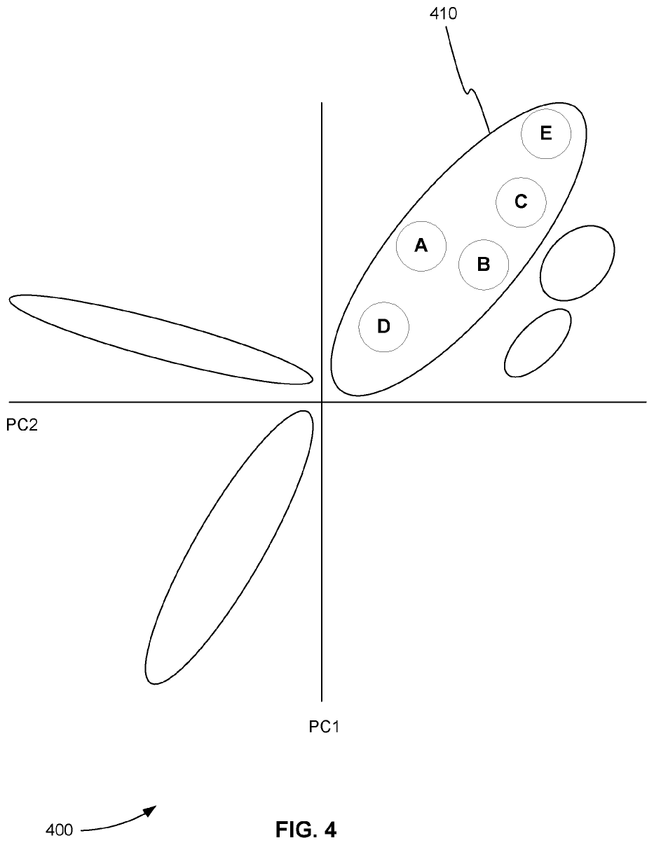

FIG. 5 is an exemplary K-means clustering algorithm plot showing the location of a protein group that includes the proteins of FIGS. 2 and 4 in relation to other protein groups or clusters, upon which embodiments of the present teachings may be implemented.

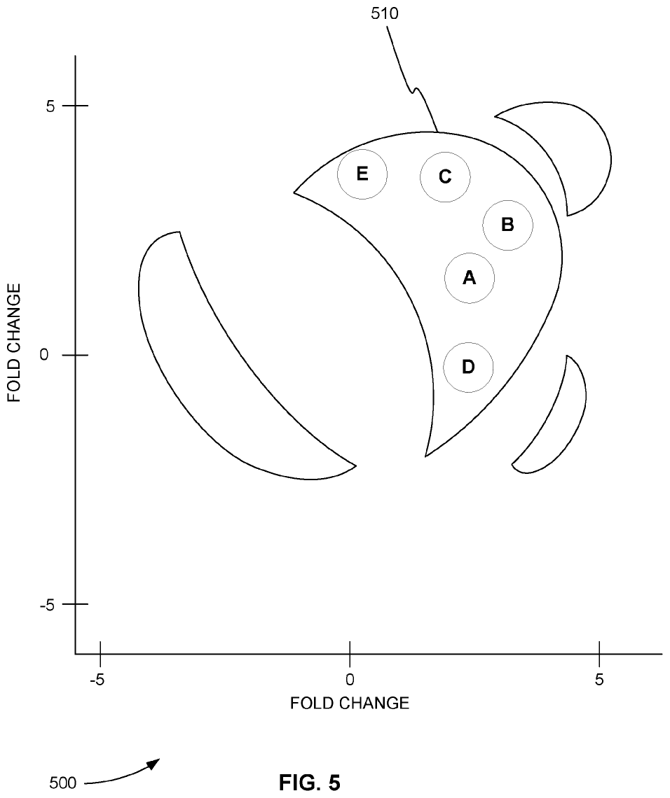

FIG. 6 is an exemplary diagram showing that the intersection of a group from the clustering algorithm of FIG. 4 and a group from the clustering algorithm of FIG. 5 produces a co-clustered protein group that includes the proteins of the biological pathway of FIGS. 2, upon which embodiments of the present teachings may be implemented.

FIG. 7 is an exemplary diagram showing a biological pathway that includes six proteins, in accordance with various embodiments.

FIG. 8 is an exemplary PCA-PCVG plot showing that reanalysis of the output from the PCA-PCVG clustering algorithm reveals the location of protein F in a separate cluster from proteins A, B, C, D, and E, in accordance with various embodiments.

FIG. 9 is an exemplary K-means clustering algorithm plot showing that reanalysis of the output from the K-means clustering algorithm reveals the location of protein F in a separate cluster from proteins A, B, C, D, and E, in accordance with various embodiments.

FIG. 10 is an exemplary co-occurrence matrix showing the co-occurrence of proteins A, B, C, D, E, and F across samples for two different clustering algorithms, in accordance with various embodiments.

FIG. 11 is an exemplary co-occurrence matrix showing the co-occurrence of proteins A, B, C, D, E, and F across samples for two different clustering algorithms that would not produce a successful verification, in accordance with various embodiments.

FIG. 12 is a schematic diagram showing a system for verifying compounds of a group detected by co-clustering are related to a biological process, in accordance with various embodiments.

FIG. 13 is a flowchart showing a method for verifying compounds of a group detected by co-clustering are related to a biological process, in accordance with various embodiments.

FIG. 14 is a schematic diagram of a system 1400 that includes one or more distinct software modules that performs a method for verifying compounds of a group detected by co-clustering are related to a biological process, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

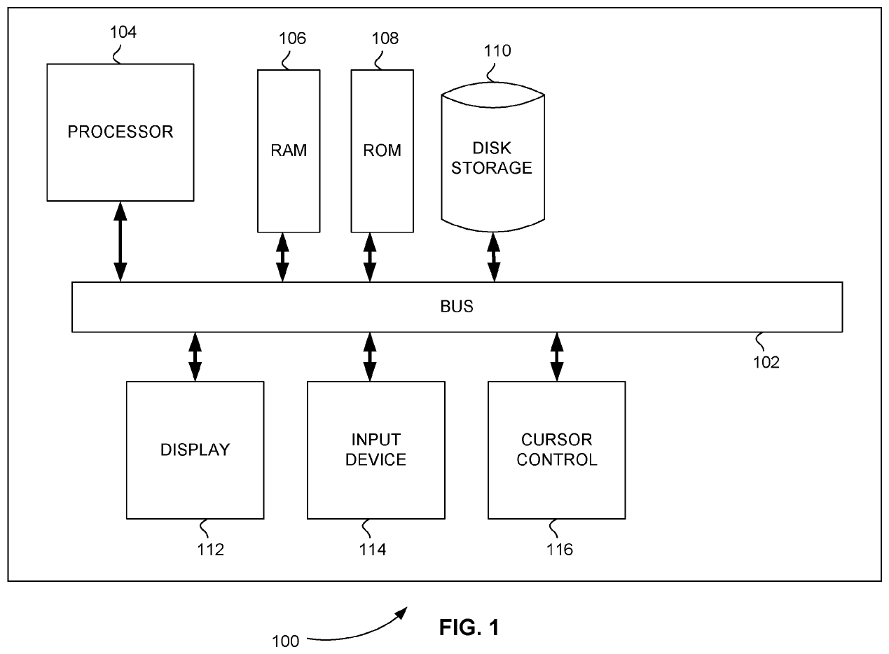

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein. Alternatively, hard-wired circuitry may be used in place of or in combination with software instructions to implement the present teachings. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media, volatile media, and precursor ion mass selection media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Verifying Co-Clustering Results

As described above, processing mass spectrometry data for biological pathways or disease indicators typically starts with the identification and then quantification of a large number of different compounds. For some time, clustering algorithms have been used to find the relationships between compounds, such as proteins, from these mass spectrometry measurements. These relationships are then compared to known interacting groups, pathways, or disease indicators to determine if a biological process was detected.

The Ronan Paper has identified three problems with the use of conventional clustering algorithms, such as PCA-PCVG and KR-means. The first problem is the high dimensionality of the data used to identify a biological pathway or disease indicator. The second problem is the failure to consider results from more than one clustering algorithm. The third problem is the difficulty in determining whether clustering or co-clustering produces meaningful results.

In regard to the third problem, the Ronan paper describes that most successful analyses often result from combining clustering with previous biological understanding of the system. The Ronan paper, however, does not describe how previous biological understanding of the system should be combined with clustering. Instead, it simply suggests that the process of attaching biological meaning to clusters be done with statistical analysis.

As a result, additional systems and methods are needed for combining clustering and co-clustering with additional biological information in order to improve the identification of biological pathways or disease indicators from mass spectrometry data.

In various embodiments, the method used in the Ronan Paper is reversed in order to improve or verify the identification of biological pathways or disease indicators. In other words, after identifying a co-clustered group of proteins using the method of the Ronan Paper and comparing that group to a biological pathway, one or more additional proteins in the biological pathway not identified by the group are used to drive a reanalysis or re-extraction of the data in order to improve or verify the identification.

For example, the use of the stable clusters, which are generated from the analysis of the combined data, produces a result that can then be clustered further to identify features that are present within the same cluster from the multitude of different cluster methods. The final clustering of this co-occurrence matrix provides an avenue to test the different components and the similarity of how they vary with the experimental data. Using a DIA method, of the mass spectrometry data does not require the reacquisition of any samples.

Specifically, for example, a list of known proteins or metabolites that belong to a biological system can be used to determine the maximum distance on a cluster dendrogram. This distance is a measure of how similar they are clustered across all of the different methods that were applied. This distance measurement is not limited to pathways or interaction networks but to any form of grouping that can be applied to the data. In various embodiments, a scoring that is generated by the distance when coupled with the fold change results provides a key insight into the changing biology which is happening in the experimental set.

In other words, in various embodiments, one or more additional compounds found in an interacting group of a cell, a biological pathway, or a disease indicator are used to improve or verify the identification of a stable group of proteins.

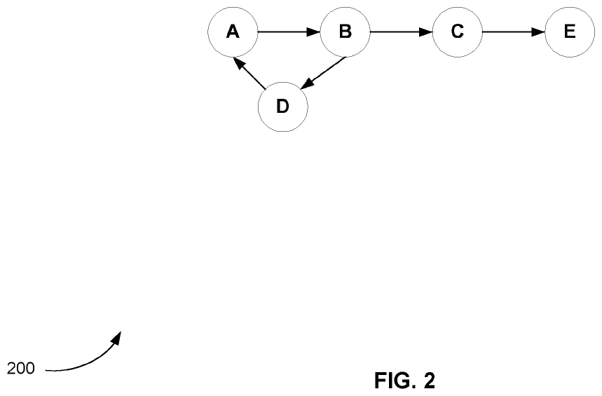

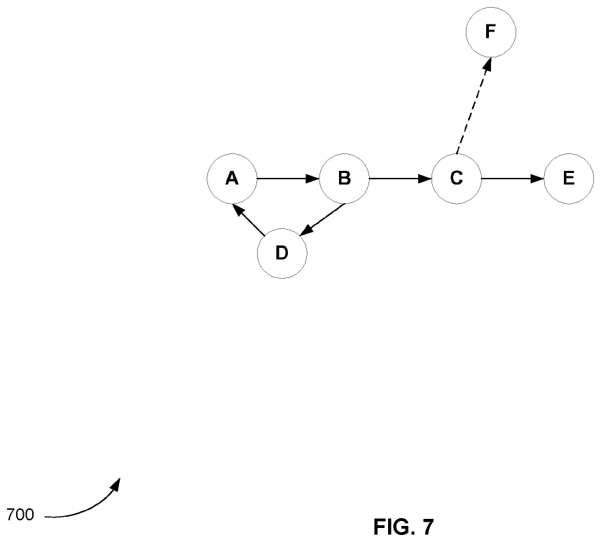

FIG. 7 is an exemplary diagram 700 showing a biological pathway that includes six proteins, in accordance with various embodiments. In comparison to the biological pathway of FIG. 2, the biological pathway of FIG. 7 includes additional protein F. Protein C links to protein F, for example.

If after analysis of a certain set of mass spectrometry data using two or more clustering algorithms, a co-clustered protein group is found, then this group is compared to one or more pathways. For example, when co-clustered protein group 610, which includes proteins A, B, C, D, and E, is found using PCA-PCVG and K-means algorithms, this group is compared to one or more pathways. This group is considered to be a stable group because all five proteins are found in the same group in each of the two different clustering algorithms.

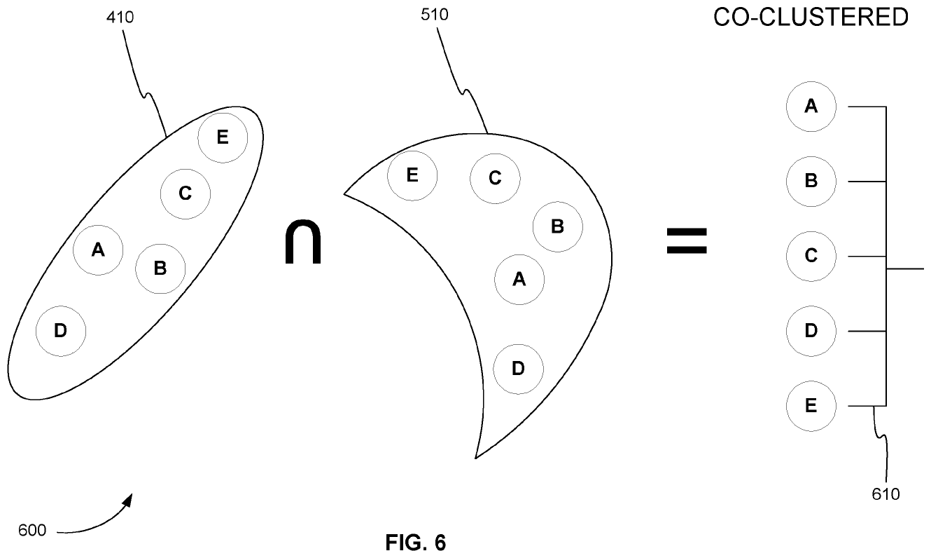

Proteins of a stable group are then compared to one or more interacting groups of a cell, one or more biological pathways, or one or more disease indicators. For example, proteins A, B, C, D, and E of stable group 610 of FIG. 6 are compared to the pathway of FIG. 7.

From this comparison, it is determined that the pathway of FIG. 7 includes at least one additional protein, F, that is not part of stable group 610 of FIG. 6. This additional protein is then used to improve or verify the identification of proteins A, B, C, D, and E as a stable group.

In various embodiments, this is done by re-analyzing the output from the two clustering algorithms to determine if protein F has been found in any clusters. If protein F was not included in the library or database used to identify proteins A, B, C, D, and E, the mass spectrometry data can be reanalyzed to identify protein F. This reanalysis can take place without having to reacquire the samples used if a DIA method is used. Because a DIA method obtains a complete record of the product ions produced across an entire mass range, reacquisition of the samples is not required to analyze any product ions not previously analyzed and identify the protein from those product ions obtains.

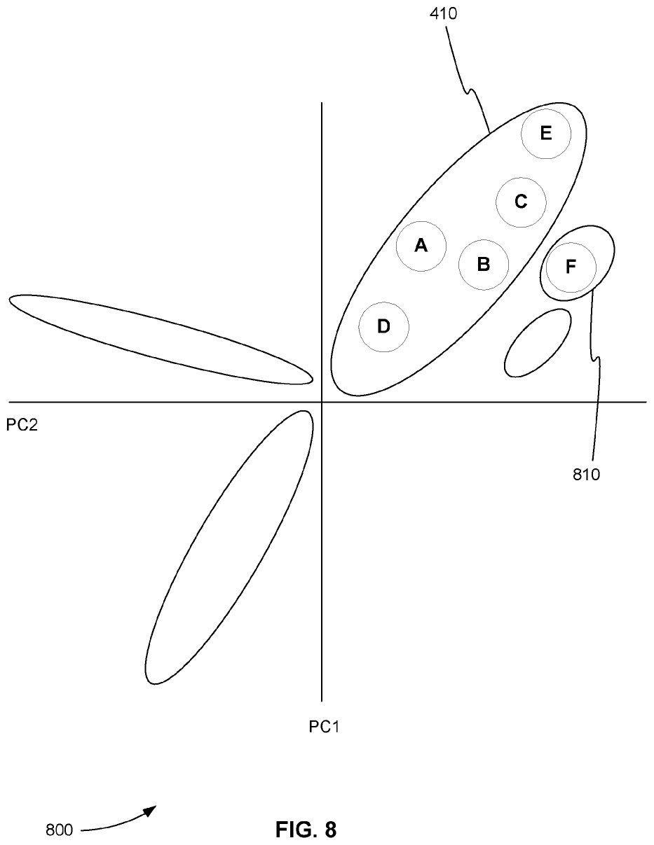

FIG. 8 is an exemplary PCA-PCVG plot 800 showing that reanalysis of the output from the PCA-PCVG clustering algorithm reveals the location of protein F in a separate cluster from proteins A, B, C, D, and E, in accordance with various embodiments. In plot 800, cluster 810 includes protein F and is separate from cluster 410, which includes proteins A, B, C, D, and E.

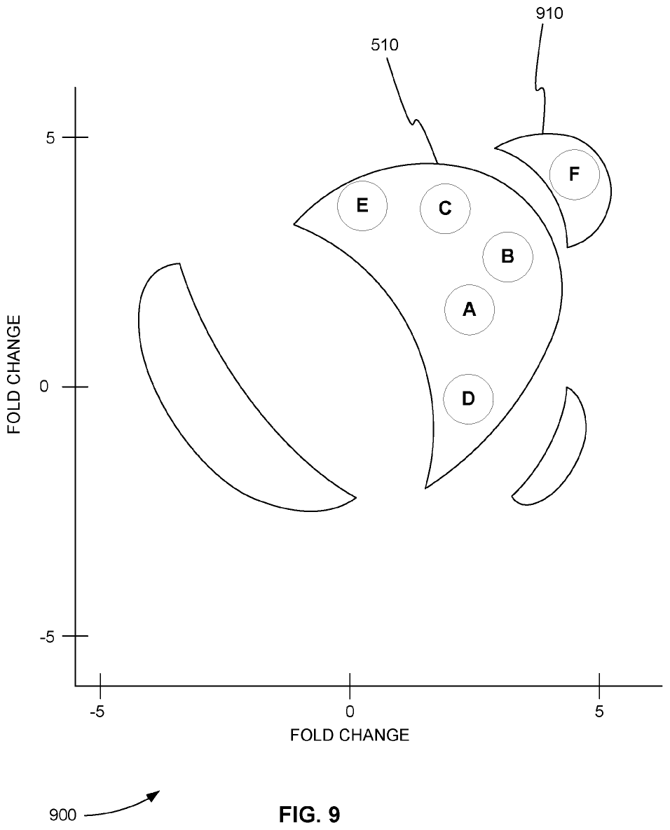

FIG. 9 is an exemplary K-means clustering algorithm plot 900 showing that reanalysis of the output from the K-means clustering algorithm reveals the location of protein F in a separate cluster from proteins A, B, C, D, and E, in accordance with various embodiments. In plot 900, cluster 910 includes protein F and is separate from cluster 510, which includes proteins A, B, C, D, and E.

Because protein F is in a separate cluster from proteins A, B, C, D, and E in the results from both clustering algorithms, a co-occurrence matrix is calculated to assess the relationship between protein F and proteins A, B, C, D, and E across the samples analyzed. Co-occurrence of protein F with proteins A, B, C, D, and E above a certain percentage of samples indicates that the pathway of FIG. 7 has been found. It also verifies that the group including proteins A, B, C, D, and E is a stable group.

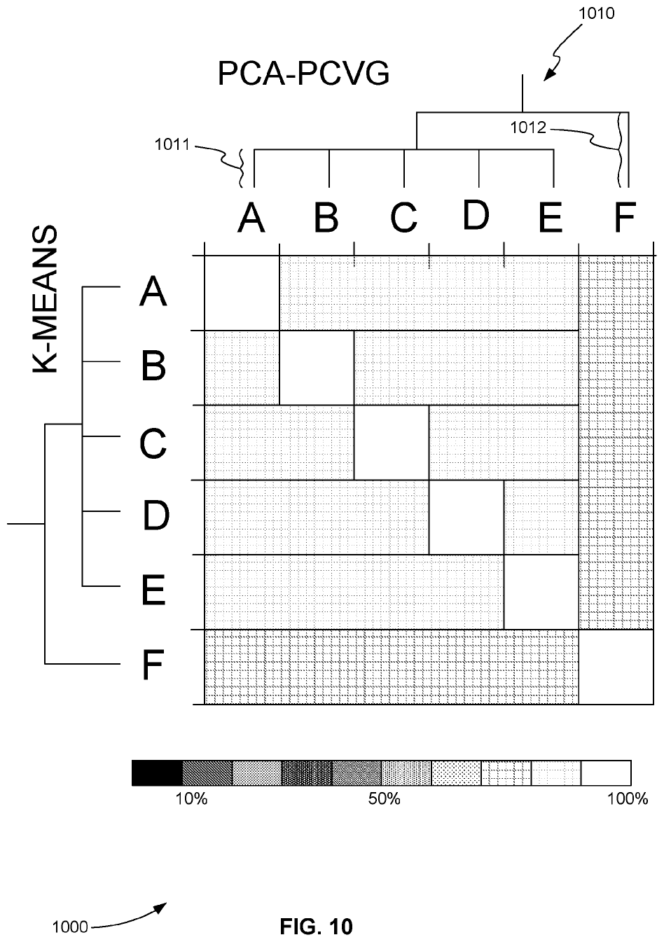

FIG. 10 is an exemplary co-occurrence matrix 1000 showing the co-occurrence of proteins A, B, C, D, E, and F across samples for two different clustering algorithms, in accordance with various embodiments. In matrix 1000, elements above the diagonal represent the percentage co-occurrence of two proteins in the samples analyzed using the PCA-PCVG clustering algorithm. Similarly, in matrix 1000, elements below the diagonal represent the percentage co-occurrence of two proteins in the samples analyzed using the K-means algorithm.

From co-occurrence matrix 1000 the distance between proteins is also determined. For the PCA-PCVG clustering algorithm, each protein of A, B, C, D, and E occurs with another protein of A, B, C, D, and E 80-90% of the time. This co-occurrence is expressed as a distance in dendrogram 1010. A distance is, for example, calculated in the space of the clustering algorithm used. The space of the PCA-PCVG clustering algorithm is a PC space. In this space, each protein of A, B, C, D, and E is one level apart from another protein of A, B, C, D, and E. In other words, the distance is one level in dendrogram 1010. Distance 1011 in FIG. 10 is one level, for example.

This distance can also be expressed in terms of the co-occurrence of percentage. In that case, each protein of A, B, C, D, and E is about 10% apart from another protein of A, B, C, D, and E in terms of occurrence. This means, for example, that when protein A occurs, protein B occurs about 90% of the time.

As shown in FIG. 8, protein F is in a cluster 810 that is different from cluster 410, which includes proteins A, B, C, D, and E. As a result, protein F is less related to proteins A, B, C, D, and E. Protein F occurs with another protein of A, B, C, D, and E 70-80% of the time.

Returning to FIG. 10, this more distant relationship between protein F and proteins A, B, C, D, and E is also shown in matrix 1000. This co-occurrence is also expressed as a larger distance in dendrogram 1010. For example, protein F is two levels apart from another protein of A, B, C, D, and E. In other words, the distance is two levels in dendrogram 1010. Distance 1012 in FIG. 10 is two levels.

Similarly, this distance can also be expressed in terms of the co-occurrence of percentage. In that case, protein F is about 20% apart from a protein of A, B, C, D, and E in terms of occurrence. This means, for example, that when protein A occurs, protein F occurs about 80% of the time.

The elements of co-occurrence matrix 1000 below the diagonal are symmetric with the elements above the diagonal. This means that, for the K-means clustering algorithm, all of the distances between proteins A, B, C, D, E, and F are the same. As a result, matrix 1000 shows that both clustering algorithms not only confirm the presence of protein F but show the same relationship between protein F and proteins A, B, C, D, and E. Consequently, reanalysis of proteins A, B, C, D, and E with protein F has reconfirmed or verified the identification of proteins A, B, C, D, and E as a stable group. It is important to note that this verification was performed even using a protein that was less related to proteins A, B, C, D, and E.

Thus, matrix 1000 shows that one or more additional compounds found in an interacting group of a cell, a biological pathway, or a disease indicator can be used to improve or verify the identification of a stable group of proteins. Although the Ronan Paper did not describe or suggest such a use for these compounds, the various embodiments described herein directly address the third problem outline in the Ronan Paper. This problem is the difficulty in determining whether clustering or co-clustering produces meaningful results.

Reanalysis of proteins of a stable cluster using one or more additional proteins from the pathway identified, however, may not always produce a successful verification. In other words, this reanalysis may sometimes determine that clustering or co-clustering produces results that are not meaningful.

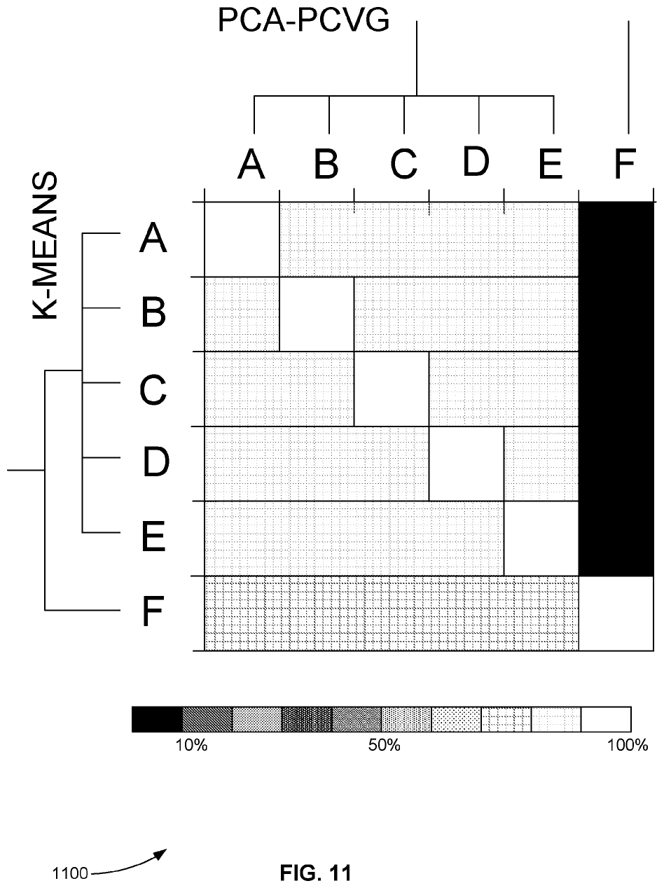

FIG. 11 is an exemplary co-occurrence matrix 1100 showing the co-occurrence of proteins A, B, C, D, E, and F across samples for two different clustering algorithms that would not produce a successful verification, in accordance with various embodiments. Co-occurrence matrix 1100 shows the same co-occurrence elements for the K-means clustering algorithm as the matrix of FIG. 10.

However, for the PCA-PCVG clustering algorithm, matrix 1100 shows that protein F occurs with each protein of A, B, C, D, and E 0% of the time. In other words, the PCA-PCVG clustering algorithm fails to show the same co-occurrence produced by the K-means clustering algorithm. In this case, proteins A, B, C, D, and E are not confirmed as a stable group. This case also shows the value of employing more than one clustering algorithm. Essentially, a false positive is prevented by using two different clustering algorithms.

FIGS. 10 and 11 show different dendrograms for the two different clustering algorithms that can have different distances. In various embodiments, the distances calculated for the two different clustering algorithms can be combined into a single distance. The distances calculated for the two different clustering algorithms can be, for example, the maximum distances calculated for the two different clustering algorithms. Similarly, the two different dendrograms of the two different clustering algorithms can be combined into a single dendrogram representing the co-clustering.

### System for Verifying Stable Clusters

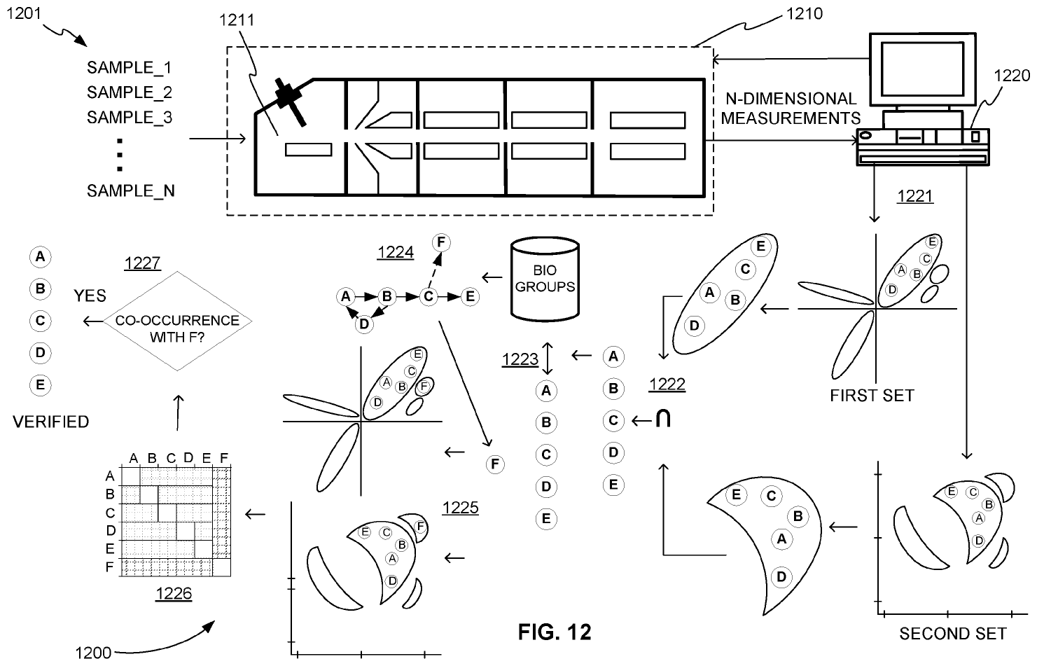

FIG. 12 is a schematic diagram 1200 showing a system for verifying compounds of a group detected by co-clustering are related to a biological process, in accordance with various embodiments. The system of FIG. 12 includes mass spectrometer 1210 and processor 1220.

Mass spectrometer 1210 fragments and mass analyzes a mass range using at least m measurements for each sample of at least n different samples 1201, producing at least m measurements in n dimensions. In various embodiments, mass spectrometer 1210 further fragments and mass analyzes the mass range using at least m measurements at each time step of at least t time steps for each sample of at least n different samples 1201, producing at least m×t measurements in n dimensions s for each sample.

Mass spectrometer 1210 is shown in FIG. 12 as a triple quadrupole device. One of ordinary skill in the art can appreciate that mass spectrometer 1210 can include other types of mass spectrometry devices including, but not limited to, ion traps, orbitraps, time-of-flight (TOF) devices, QqLIT devices, or Fourier transform ion cyclotron resonance (FT-ICR) devices.

In various embodiments, the system of FIG. 12 further includes a sample introduction device (not shown) and ion source device 1211. The sample introduction device introduces each sample of the at least n different samples 1201 to the system over time. A sample is obtained from a sample plate, for example. Sample introduction device 1210 can perform techniques that include, but are not limited to, ion mobility, gas chromatography (GC), liquid chromatography (LC), capillary electrophoresis (CE), acoustic ejection mass spectrometry (AEMS), or flow injection analysis (FIA).

Ion source device 1211 ionizes compounds of a sample to transform the compounds into an ion beam. Ion source device 1211 can perform ionization techniques that include, but are not limited to, matrix-assisted laser desorption/ionization (MALDI) or electrospray ionization (ESI).

Processor 1220 can be, but is not limited to, a computer, a microprocessor, the computer system of FIG. 1, or any device capable of sending and receiving control signals and data from mass spectrometer 1210 and processing data. Processor 1220 is in communication with mass spectrometer 1210.

Processor 1220 receives the n-dimensional measurements from mass spectrometer 1210. In step 1221, processor 1220 applies a first clustering algorithm and a second clustering algorithm to the n-dimensional measurements, producing a first set of clusters and a second set of clusters, respectively, and identifies compounds in the first set and the second set. In step 1222, processor 1220 selects a first cluster from the first set and a second cluster from the second set that includes the same two or more compounds.

In step 1223, processor 1220 compares the two or more compounds to one or more groups of compounds related to a biological process to identify at least one group of the one or more groups that includes the two or more compounds. In step 1224, processor 1220 selects at least one compound other than the two or more compounds in the at least one group. In step 1225, processor 1220 reanalyzes the first set and the second set to identify the at least one compound in the first set and the second set.

In step 1226, processor 1220 calculates a co-occurrence matrix that quantifies the co-occurrence of the at least one compound and each of the two or more compounds for both the first set and the second set. In step 1227, if no co-occurrence quantity of the at least one compound and each of the two or more compounds in the co-occurrence matrix is below a predetermined threshold, processor 1220 verifies that the two or more compounds are related to a biological process.

In various embodiments, processor 1220 identifies compounds in the first set and the second set by comparing measurements in the first set and the second set to a library of measurements for known compounds. In alternative various embodiments, processor 1220 identifies compounds in the first set and the second set by comparing measurements in the first set and the second set to compound fragments predicted from a compound database.

In various embodiments, the first clustering algorithm includes PCA-PCVG and the second clustering algorithm includes K-means. In various alternative embodiments, the first clustering algorithm and the second clustering algorithm can be any two different clusters algorithms.

In various embodiments, a compound can be a protein or a metabolite.

In various embodiments, the one or more groups of compounds related to a biological process include one or more interacting groups of compounds of a cell, one or more biological pathways, or one or more disease indicators. The one or more interacting groups of compounds of a cell, one or more biological pathways, or one or more disease indicators are obtained from a database, for example.

In various embodiments, mass spectrometer 1210 fragments and mass analyzes the mass range using a DIA method. The DIA method allows processor 1220 to reanalyze the first set and the second set to identify the at least one compound in the first set and the second set without having to reacquire at least n different samples 1201.

In various embodiments, processor 1220 quantifies the co-occurrence of the at least one compound and each of the two or more compounds for both the first set and the second set by calculating a percentage of co-occurrence across the n samples for the at least one compound and each of the two or more compounds for both the first set and the second set. The predetermined threshold is then a percentage of co-occurrence threshold. In various alternative embodiments, processor 1220 quantifies the co-occurrence of the at least one compound and each of the two or more compounds for both the first set and the second set by calculating distance in the space of the clustering algorithm used for the at least one compound and each of the two or more compounds for both the first set and the second set. The predetermined threshold is then a distance in the space of the clustering algorithm used.

### Method for Verifying Stable Clusters

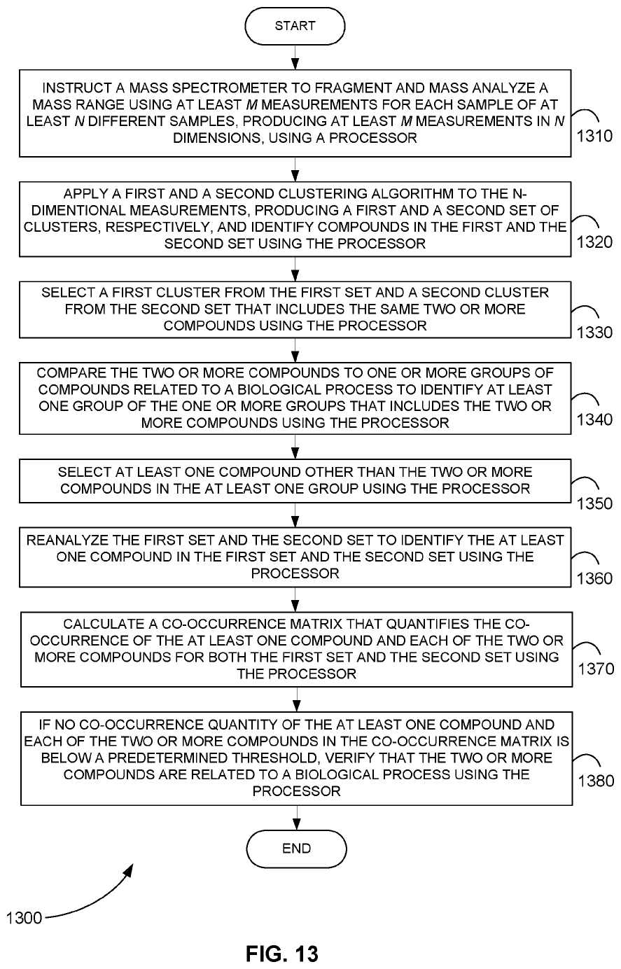

FIG. 13 is a flowchart showing a method 1300 for verifying compounds of a group detected by co-clustering are related to a biological process, in accordance with various embodiments.

In step 1310 of method 1300, a mass spectrometer is instructed to fragment and mass analyze a mass range using at least m measurements for each sample of at least n different samples, producing at least m measurements in n dimensions, using a processor.

In step 1320, a first clustering algorithm and a second clustering algorithm are applied to the n-dimensional measurements, producing a first set of clusters and a second set of clusters, respectively, and compounds in the first set and the second set are identified using the processor.

In step 1330, a first cluster is selected from the first set and a second cluster is selected from the second set that includes the same two or more compounds using the processor.

In step 1340, the two or more compounds are compared to one or more groups of compounds related to a biological process to identify at least one group of the one or more groups that includes the two or more compounds using the processor.

In step 1350, at least one compound other than the two or more compounds is selected in the at least one group using the processor.

In step 1360, the first set and the second set are reanalyzed to identify the at least one compound in the first set and the second set using the processor.

In step 1370, a co-occurrence matrix is calculated that quantifies the co-occurrence of the at least one compound and each of the two or more compounds for both the first set and the second set using the processor.

In step 1380, if no co-occurrence quantity of the at least one compound and each of the two or more compounds in the co-occurrence matrix is below a predetermined threshold, the two or more compounds are verified as being are related to a biological process using the processor.

### Computer Program Product for Verifying Stable Clusters

In various embodiments, a computer program product includes a non-transitory tangible computer-readable storage medium whose contents include a program with instructions being executed on a processor so as to perform a method for verifying compounds of a group detected by co-clustering are related to a biological process. This method is performed by a system that includes one or more distinct software modules.

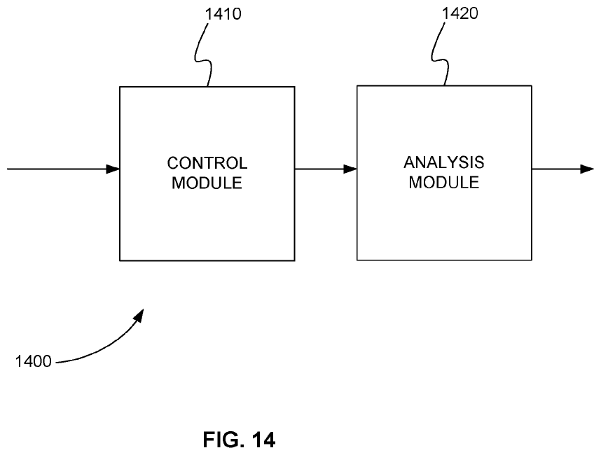

FIG. 14 is a schematic diagram of a system 1400 that includes one or more distinct software modules that performs a method for verifying compounds of a group detected by co-clustering are related to a biological process, in accordance with various embodiments. System 1400 includes control module 1410 and analysis module 1420.

Control module 1410 instructs a mass spectrometer to fragment and mass analyze a mass range using at least m measurements for each sample of at least n different samples, producing at least m measurements in n dimensions.

Analysis module 1420 applies a first clustering algorithm and a second clustering algorithm to the n-dimensional measurements, producing a first set of clusters and a second set of clusters, respectively, and identifies compounds in the first set and the second set. Analysis module 1420 selects a first cluster from the first set and a second cluster from the second set that includes the same two or more compounds. Analysis module 1420 compares the two or more compounds to one or more groups of compounds related to a biological process to identify at least one group of the one or more groups that includes the two or more compounds.

Analysis module 1420 selects at least one compound other than the two or more compounds in the at least one group. Analysis module 1420 reanalyzes the first set and the second set are to identify the at least one compound in the first set and the second set. Analysis module 1420 calculates a co-occurrence matrix that quantifies the co-occurrence of the at least one compound and each of the two or more compounds for both the first set and the second set. If no co-occurrence quantity of the at least one compound and each of the two or more compounds in the co-occurrence matrix is below a predetermined threshold, analysis module 1420 verifies that the two or more compounds are related to a biological process using the processor.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A system for verifying compounds of a group detected by co-clustering are related to a biological process, comprising:
a mass spectrometer that is configured to fragment and mass analyze a mass range using at least m measurements for each sample of at least n different samples, producing at least m measurements in n dimensions; and
a processor configured to:
apply a first and a second clustering algorithm to the n-dimensional measurements, produce a first and a second set of clusters, respectively, and identify compounds in the first and the second set,
select a first cluster from the first set and a second cluster from the second set that includes the same two or more compounds,
compare the two or more compounds to one or more groups of compounds related to a biological process to identify at least one group of the one or more groups that includes the two or more compounds,
select at least one compound other than the two or more compounds in the at least one group,
identify the at least one compound in the first set and the second set,
calculate a co-occurrence matrix that quantifies co-occurrence of the at least one compound and each of the two or more compounds for both the first set and the second set, and
verify that the two or more compounds are related to a biological process if no co-occurrence quantity of the at least one compound and each of the two or more compounds in the co-occurrence matrix is below a predetermined threshold.

2. The system of claim 1, wherein the processor is configured to identify compounds in the first and the second set by comparing measurements in the first and the second to a library of measurements for known compounds.

3. The system of claim 1, wherein the processor is configured to identify compounds in the first and the second set by comparing measurements in the first and the second set to compound fragments predicted from a compound database.

4. The system of claim 1, wherein the first clustering algorithm comprises principal component analysis with principal component variable grouping (PCA-PCVG).

5. The system of claim 4, wherein the second clustering algorithm comprises K-means.

6. The system of claim 1, wherein the mass spectrometer is further configured to fragment and mass analyze the mass range using at least m measurements at each time step of at least t time steps for each sample of at least n different samples, producing at least m×t measurements in n dimensions for each sample.

7. The system of claim 1, wherein any of the compounds comprises a protein or a metabolite.

8. The system of claim 1, wherein the one or more groups of compounds related to a biological process comprise one or more interacting groups of compounds of a cell.

9. The system of claim 1, wherein the one or more groups of compounds related to a biological process are related to one or more biological pathways.

10. The system of claim 1, wherein the one or more groups of compounds related to a biological process comprise one or more disease indicators.

11. The system of claim 1, wherein the mass spectrometer is configured to fragment and mass analyze the mass range using a data independent acquisition (DIA) method.

12. The system of claim 1, wherein the processor is configured to quantify the co-occurrence of the at least one compound and each of the two or more compounds for both the first set and the second set by calculating a percentage of co-occurrence across the n samples for the at least one compound and each of the two or more compounds for both the first set and the second set and wherein the predetermined threshold is a percentage of co-occurrence.

13. The system of claim 1, wherein the processor is configured to quantify the co-occurrence of the at least one compound and each of the two or more compounds for both the first set and the second set by calculating distance in the space of the clustering algorithm used for the at least one compound and each of the two or more compounds for both the first set and the second set and wherein the predetermined threshold is a distance in the space of the clustering algorithm used.

14. A method for verifying compounds of a group detected by co-clustering are related to a biological process, comprising:
instructing a mass spectrometer to fragment and mass analyze a mass range using at least m measurements for each sample of at least n different samples, producing at least m measurements in n dimensions, using a processor;
applying a first and a second clustering algorithm to the n-dimensional measurements, producing a first and a second set of clusters, respectively, and identifying compounds in the first and the second set using the processor;
selecting a first cluster from the first set and a second cluster from the second set that includes the same two or more compounds using the processor;
comparing the two or more compounds to one or more groups of compounds related to a biological process to identify at least one group of the one or more groups that includes the two or more compounds using the processor;
selecting at least one compound other than the two or more compounds in the at least one group using the processor;
identifying the at least one compound in the first set and the second set using the processor;
calculating a co-occurrence matrix that quantifies the co-occurrence of the at least one compound and each of the two or more compounds for both the first set and the second set using the processor; and
verifying that the two or more compounds are related to a biological process if no co-occurrence quantity of the at least one compound and each of the two or more compounds in the co-occurrence matrix is below a predetermined threshold.

15. A computer program product, comprising a non-transitory tangible computer-readable storage medium whose contents include a program with instructions being executed on a processor for verifying compounds of a group detected by co-clustering are related to a biological process, comprising:
providing a system, wherein the system comprises one or more distinct software modules, and wherein the distinct software modules comprise a control module and an analysis module;
instructing a mass spectrometer to fragment and mass analyze a mass range using at least m measurements for each sample of at least n different samples, producing at least m measurements in n dimensions, using the control module;
applying a first and a second clustering algorithm to the n-dimensional measurements, producing a first and a second set of clusters, respectively, and identifying compounds in the first and the second set using the processor;
selecting a first cluster from the first set and a second cluster from the second set that includes the same two or more compounds using the analysis module;
comparing the two or more compounds to one or more groups of compounds related to a biological process to identify at least one group of the one or more groups that includes the two or more compounds using the analysis module;
selecting at least one compound other than the two or more compounds in the at least one group using the analysis module;
identifying the at least one compound in the first set and the second set using the analysis module;
calculating a co-occurrence matrix that quantifies the co-occurrence of the at least one compound and each of the two or more compounds for both the first set and the second set using the analysis module; and
using the analysis module to verify that the two or more compounds are related to a biological process using if no co-occurrence quantity of the at least one compound and each of the two or more compounds in the co-occurrence matrix is below a predetermined threshold.

16. The system of claim 1, wherein the processor is configured to reanalyze the first set and the second set to identify the at least one compound in the first set and the second set.

17. The method of claim 14, wherein the step of identifying the at least one compound in the first set and the second set comprises reanalyzing the first and the second set.

18. The computer program product of claim 15, wherein the step of identifying the at least one compound in the first set and the set comprises using the analysis module to reanalyze the first set and the second set.
